(19) United States
(12) Patent Application Publication
(10) Pub. No.: US 2022/0395425 A1
(43) Pub. Date: Dec. 15, 2022

(54) THERMAL ELECTRONIC MASSAGE NEUROMUSCULAR ROLLER AND VIBRATOR

(71) Applicant: Christopher Diecidue, Chandler, AZ (US)

(72) Inventor: Christopher Diecidue, Chandler, AZ (US)

(21) Appl. No.: 17/344,493

(22) Filed: Jun. 10, 2021

(51) Int. Cl. A61H 15/02; A61H 15/00

(52) U.S. Cl. CPC A61H 15/02; A61H 15/0092; A61H 2015/0014; A61H 2201/0207; A61H 2201/0228; A61H 2201/5025

(57) ABSTRACT

A multi-layer thermal electronic massage roller with a cylindrical body forming a generally solid body, including an innermost core layer, insulative layer, heating element binder layer, a heating element, a conductive sleeve to pull heat away from the electronic components and direct it to the user, a stiff cylindrical layer, and an outermost fabric layer. The device further includes a battery, a power port and a thermal control module to variably adjust the temperature. A method for configuring the multi-layer thermal electronic massage roller is also provided, including configuration of the elements of the device for optimum performance and disclosure of materials for embodiments of the invention.

### FIELD OF THE INVENTION

This invention relates to massage rollers and massage equipment.

### BACKGROUND OF THE INVENTION

This invention is an improvement of the inventor's patent application, publication US 2014/0371639 (2014).

Massage has long been used to treat tight, stiff, and injured muscles, having benefits such as prevention and prediction of muscle injuries. Massage can improve strength, flexibility, endurance, dispersal of lactic acid, and accelerate recovery of numerous physical conditions. Athletes and exercise aficionados have long turned to massage to improve performance and ease aching muscles. Using a licensed massage therapist is beneficial, but easily becomes expensive.

Additionally, there are times when immediate treatment is necessary. Therefore, hand held massage devices were developed to address these needs. Handheld massage devices are also often used to treat trigger points. One type of handheld massage device is a wand that can be rolled over a user's muscles to provide relief. The massage wand can include a roller or rollers over its outer surface. Unfortunately, these devices do not offer any variety in roller design unless a user buys multiple products, and therefore, cannot treat an array of muscle problems or with a single device.

The device constitutes an improvement over standard massage devices. Some massage devices primarily include a unitary object that one may grip for use by a massage therapist in deep tissue massage such as shiatsu, Bowen therapy, Balinese massage and the like. Massage devices of these types serve the common goals of relieving muscle aches and sore muscles, promoting blood circulation, relieving muscle cramps, loosening tight and knotted muscles, and relieving pressure upon the message therapist's hands. These devices run the gamut from the pulling and pushing of tissues to a spectrum of articles including objects as simple as heated stones to as complex as hand held tools with complexly formed protrusions for the kneading of facia and other such connective tissue. These objects are typically not intended for self-use by a person suffering from sore muscles, joints or the like, and often only contact small surface areas of tissue and are not adapted for the application of pressure to multiple sites or across a larger surface area that may require attention.

Thus, a need has arisen in the field of massage therapy, and personal wellness devices that overcome the shortcomings of traditional devices on the market, and provide a multi-use device to aid in the relaxation, massage, and recovery processes.

### SUMMARY OF THE INVENTION

Provided is a thermal electronic massage roller, comprised of a device with a cylindrical body comprising at least six layers, wherein said at least six layers contact one another and form a generally solid body.

The layers include an innermost core layer formed of a substantially solid insulative material with a plurality of cavities for internal components of said device, wherein said internal components include at least a battery, a power port in electrical communication with said battery, and a thermal control module in electrical communication with said battery. The layers include an insulative layer cylindrically surrounding said innermost core layer, providing a thermal buffer between said innermost core layer and a heating element binder layer. The heating element binder layer cylindrically surrounds the insulative layer.

Further provided is a heating element set within said heating element binder layer. A conductive sleeve cylindrically surrounding said heating element binder is also provided, wherein said conductive sleeve is constructed of conductive material capable of pulling heat from the heating element in an outward direction away from the core of the device.

A stiff cylindrical layer is provided, surrounding the conductive sleeve to provide the structure for the device. An outermost layer is provided surrounding said stiff cylindrical layer is configured to be a formable material, wherein the outermost layer is removable, and the innermost layer further includes and an electronic couple between said heating element and said battery.

The invention also provides for a method for configuring a massage roller for thermal transfer, including configuring a device in a multi-layer construction, wherein the device includes at least six layers, and configuring each layer in the at least six layers to perform a specific function, wherein an outermost layer is configured to be a removable and washable layer that comprises form-fitting properties of a layer immediately beneath the outermost layer, a layer beneath an outermost layer is configured to be a stiff cylindrical layer to give the device structure and shape, a conductive sleeve layer is configured to be beneath the stiff cylindrical layer to evenly distribute heat of a heating element, a heating element binder layer is configured to be beneath the conductive sleeve layer to contain the heating element and provide a buffer between levels beneath the heating element binder layer, configuring a layer beneath the heating element binder layer to be an insulative layer to provide a heat buffer between the heating element and a core structure containing electronic components, and configuring the core structure as an innermost layer to contain the electronic components and act as one final heat buffer between the heating element and the electronic components, and to contain the electronic components from movement by nesting the electronic components in a solid material.

Additionally included in the method is configuring the conductive sleeve layer to draw heat away from the core by using conductive material to draw heat in a singular direction and thermally transfer the heat to a user's body by implementing a metallic matrix to act as a heat sync to transfer heat from the heating element to the user's body, and configuring the heating element for uniform heat transfer by implementing a cylindrical grid of conductive material, that, when electrically engaged, is capable of variable heat output.

It is accordingly an object of the present invention to provide a thermal massage therapy device which while providing comfort to the skin and tissue of the user to provide a beneficial degree of heat to the affected tissue.

It is a further object to provide a thermal therapy device which may operate without need to microwave any operative portion thereof before its use.

It is another object of the invention to provide a thermal massage therapy device which permits the application of considerable pressure without the risk of injury to the patient as would be the case in the absence of the foam-like surface of the present device and its associated thermal capacity.

It is a yet further object to provide a device effective in the producing of myo-fascsial release and reduction of pain.

It is another object of the invention to provide a device of the above type which can be used either by an individual or as under the direction of a qualified healthcare professional or personal trainer.

The above and yet further objects and advantages of the present invention will become apparent from the hereinafter set forth Brief Description of the Drawings, Detailed Description of the Invention and Claims appended herewith.

### BRIEF DESCRIPTION OF THE DRAWINGS

FIG. 1 is an isometric view of the thermal-electronic massage roller.

FIG. 2 is a front view of the thermal-electronic massage roller showing the variable heating control area and power port.

FIG. 3A is an enlarged isolated isometric view of the variable heating control area and power port shown in FIG. 2 with variable heat positioning shown.

FIG. 3B is an enlarged isolated isometric view of the variable heating control area and power port shown in FIG. 2 with off positioning shown.

FIG. 4 is an isometric view of the internal heating grid with electrical components.

FIG. 5 is an enlarged isometric cut-view of the internal heating grid with electrical components shown in FIG. 4.

FIG. 6 is an enlarged side cut-view of the internal heating grid with electrical components shown in FIG. 4.

FIG. 7 is an exploded view of the electrical components of the system.

FIG. 8 is an isometric cut-view of the thermal-electronic massage roller.

FIG. 9 is a side-cut view of the thermal-electronic massage roller.

FIG. 10 is an isometric side-cut view of the thermal-electronic massage roller shown in FIG. 9.

FIG. 11 is a front cut view of the thermal-electronic massage roller showing internal components.

### DETAILED DESCRIPTION OF THE APPLICATION

The present invention constitutes an improvement to standard massage devices. This device helps increase circulation to get more oxygen and nutrients to soft tissues and vital organs. Like with professional massages, regular use of this device also enhances muscle relaxation, optimizes correction of muscle imbalances, improves joint range of motion, decreases pain, and decreases muscle spasms.

The invention provides for a thermal massaging roller defined by a cylindrical body. The cylindrical body includes several layers, each of which is generally cylindrical, each with a distinct function. The device may be used as a warm-up for tissue prior to workout or activity, used as a post-work out tool to push lactic acid out of tissues and reduce recovery time. In addition, while using this device, one can expect a stimulation of the flow of lymph, the body's natural defense system, against toxic invaders. Use of this device with massage will release endorphins to control and relieve pain for chronic illness, injury, and recovery.

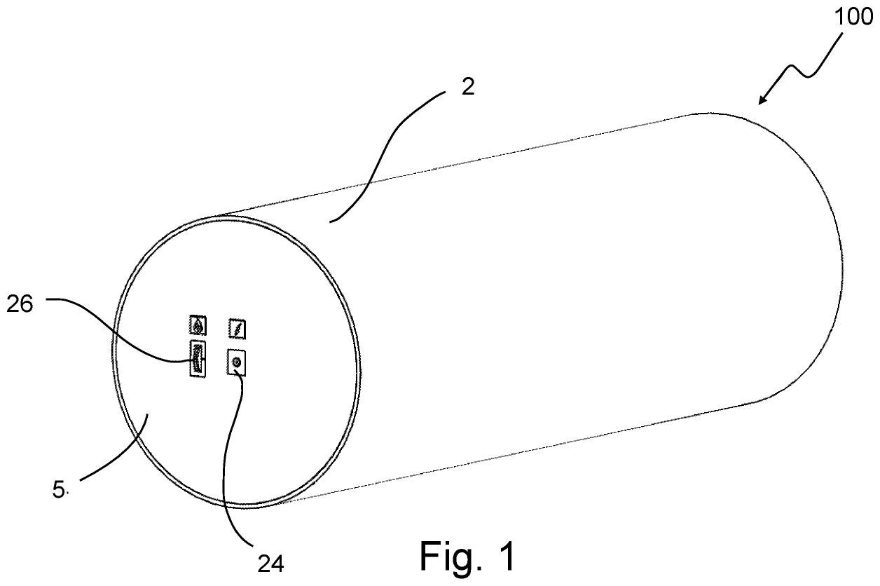

FIGS. 1-11 show an embodiment of the invention. An outer layer is defined by an outer sleeve 2 that is antimicrobial, removable, and washable. The material of this outer layer 2 is a soft fabric, which is intended to conform to the structure of the layer beneath. In some embodiments, the outer layer 2 is constructed of a material, which can be cotton cloth, polyester cloth, or rayon cloth. In addition to providing a washable surface for the device, the fabric also enhances the heat transfer from the device to a user.

The second layer 4, the layer below the outer sleeve, is the structured roller surface 4. The structured roller surface 4, like the outer layer 2, is removable. This layer may include a ribbed texture in some embodiments, but may also be smooth. The material of the structured roller may include rubber, thermoset, a dense foam of neoprene, EVA (Ethylene-vinyl acetate), or a combination of these materials. Specific material may depend on the embodiment, and the level of stiffness desired for a particular application. Further textures may also include a studded texture for massage and concentrated pressure. The optimal texture may depend on the level of pressure a user desires. This layer provides the firmness necessary for massaging, and although there is another layer over top of this layer, the texture of this layer is what the user senses while the device is being used. Because this device encapsulates electronics, the structured roller surface layer is water resilient and hydrophobic to prevent penetration of moisture into the inner layers.

Internal to the second layer is a conductive sleeve 6. The conductive sleeve 6 directs heat in a one-way direction and away from the internal system to the user. This conductive sleeve 6 helps further protect the battery 20 and electrical elements, which are susceptible to excess heat. The conductive sleeve 6 also reduces the power requirements by channeling the heat outward, because the internal layers are not receiving a heat transfer. This saves battery life. Further, by providing a singular directional heat transfer, the unit heats up quicker since it is not heating a larger volume, which allows the device to provide a more predictable and consistent supply of heat with less warm-up time. In some embodiments, the conductive sleeve 6 may be made of a matrix of metallic materials. These materials can include silver, copper, aluminum, or ferrous metals. The metallic matrix helps draw heat away from the core.

The conductive sleeve 6 acts similar the principles of a heat sink. The heat binder layer 8 keeps the heat from the heating element 12 from transferring inward. The conductive sleeve 6 pulls the heat from the heating element 12 and spreads it out evenly along the roller 100, similar to how a heat sink draws heat. The heat then transfers to the user of the massage roller 100, allowing the conductive sleeve 6 to draw more heat away from the heating element 12.

In conjunction with the conductive sleeve 6 is the heat element binder 8, wherein the heating element 12 is set inside. The heating binder layer 8 is a dense insulating retention layer. This heating binder 8 may be constructed of a dense potting material, such as silicone, epoxy, or urethane. The combination between the heating element 12, the conductive sleeve 6, and the heat element binder 8 allows the device to heat up and spread the heat around evenly, but the combination also channels the heat from the heating element through the conductive sleeve 6, while keeping the core cooler, thereby protecting the electronics therein without any need for spinning or mechanical parts, such as fans, to cool the inner cores.

Inside the heating element binder 8 is the heating element 12 itself. The heating element 12 is a mesh cylinder to evenly spread out heat to all areas of the conductive sleeve 6. The mesh cylinder 12 may be constructed of a metallic resistive conductor element; a doped ferrous metallic. The heating element 12 is powered by a rechargeable power source 20, such as a lithium-ion battery, coupled to a control board or wire junction 22 with a power port 24 and a control interface 26. The control interface 26 may be a dial with variable heating level control 30/32, a touch interface, or an interface with buttons.

The core housing 18 insulates the battery 20 from excessive heat for longevity of the system. In some embodiments, the core 18 utilizes a split-shell design that allows easy assembly of the components that are nested inside. Imprints are made for the electrical components therein to fit snugly inside the core housing 18. The imprints exist for the battery 20, splitter and/or circuitry 22, on/off regulator 26, charging port 24, and all wiring 28 therein, including wiring 28 between the components and a connecting wire 13/10 to the heating element. The core 18 may comprise moldable foam of polyurethane or silicone. Further, an insulating layer 16 may wrap around the core 18 to further provide a buffer layer of closed cell foam of polyethylene. The use of this material helps alleviate drawbacks of the prior art that result in overheating, or the need to use fans and other cooling devices, that may further draw power and reduce battery life and require servicing to keep running.

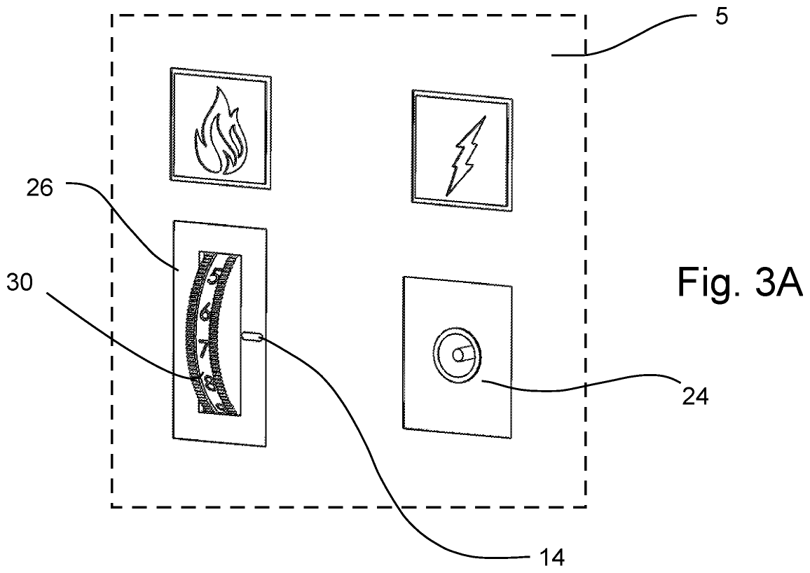

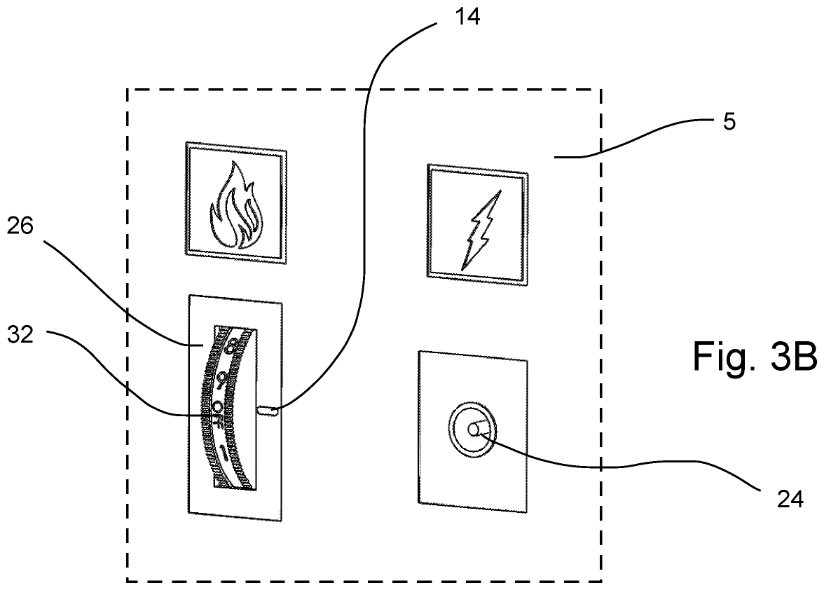

The cylinder body is capped by a pair of end caps 5. In at least one of the end caps 5, a charging port 24 and a thermal control means 26 is included, which may be either a dial, as discussed above, with multiple levels, as shown in FIGS. 3A and 3B, but may also be an on/off switch, a multi-step switch, a button, or a touch interface.

The thermal-electronic massage roller 100 is generally of a solid construction. The phrase “generally solid” means mostly solid, but with cavities 34 for components and wring 28, which, when added, will use the available space within the cavities 34. However, each layer abuts the other layers that surround it, giving it a solid feel. Moreover, this indicates that the roller 100 is not hollow, and not of a hollow construction. There are limitations to having hollow components in foam rollers, which may affect weight displacement, include moving parts or components that may be broken more easily, and, when dealing with thermal exchange, would require a cooling fan, which has numerous drawbacks as discussed above. The use of the multi-layer multi-material construction configures the massage roller to accomplish thermal exchange, but with less drawbacks than the prior art.

Further, this use of a solid construction results in less electrical components for a simpler construction and longer lifespan since the probability of parts breaking down from repeated use is reduced with the reduction of electronic components.

FIG. 1 shows is an isometric view of the thermal-electronic massage roller 100. The roller 100, as may be appreciated, is cylindrical with an outer washable sleeve 2, constructed of a flexible washable material. The roller 100 is shown having an end cap 5 on each side of the roller 100, wherein one end cap is configured to include a thermal management control 26 and a power port 24 for wired charging.

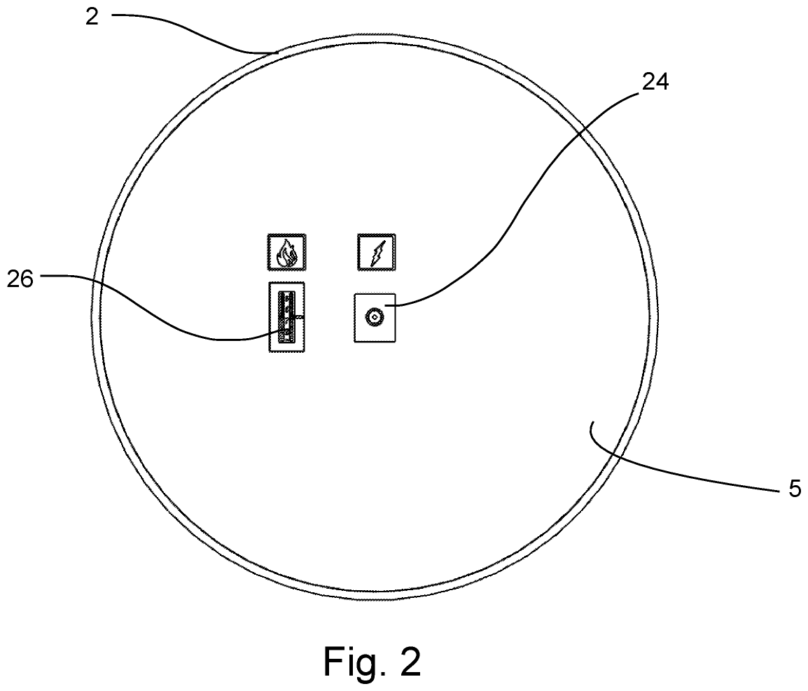

FIG. 2 is a front view of the thermal-electronic massage roller 100 showing as shown in FIG. 1. FIG. 2 shows more specifically the end cap 5 including the thermal management control 26 and power port 24. FIGS. 3A and 3B are enlarged isolated isometric views of the control area 26 and power port 24 shown in FIG. 2 with variable heat positioning shown 30/32 that illustrates the various levels of heat the roller 100 may give off. In FIG. 3A, the thermal management control 26, in the configuration of a dial, is shown with a plurality of different levels 30, and a level indicator 14 wherein a user can easily identify which level the massage roller 100 is set to. FIG. 3B shows the thermal management control 26 with the dial in its off position 32. It may be appreciated from the drawings that the dial is shown as one embodiment, but the thermal control may take other forms, such as a button that increases the heat output each time the button is pressed, or a touch screen with interface that adjusts the heating output when engaged.

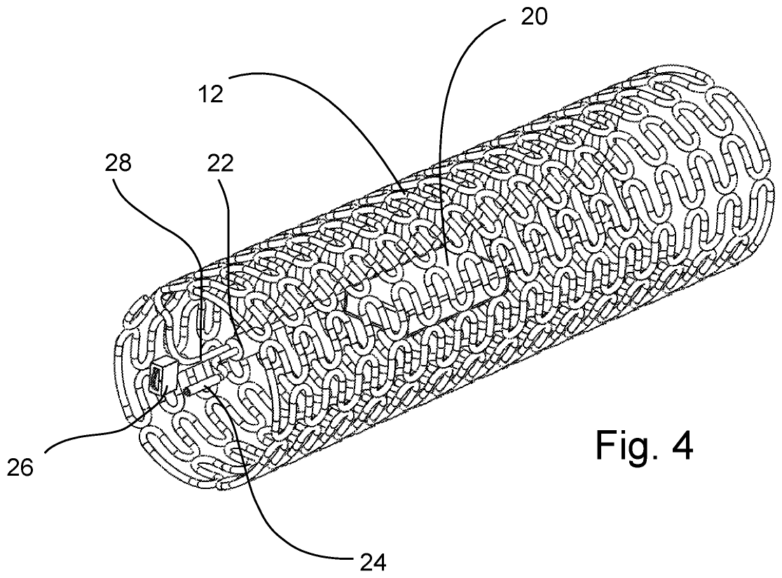

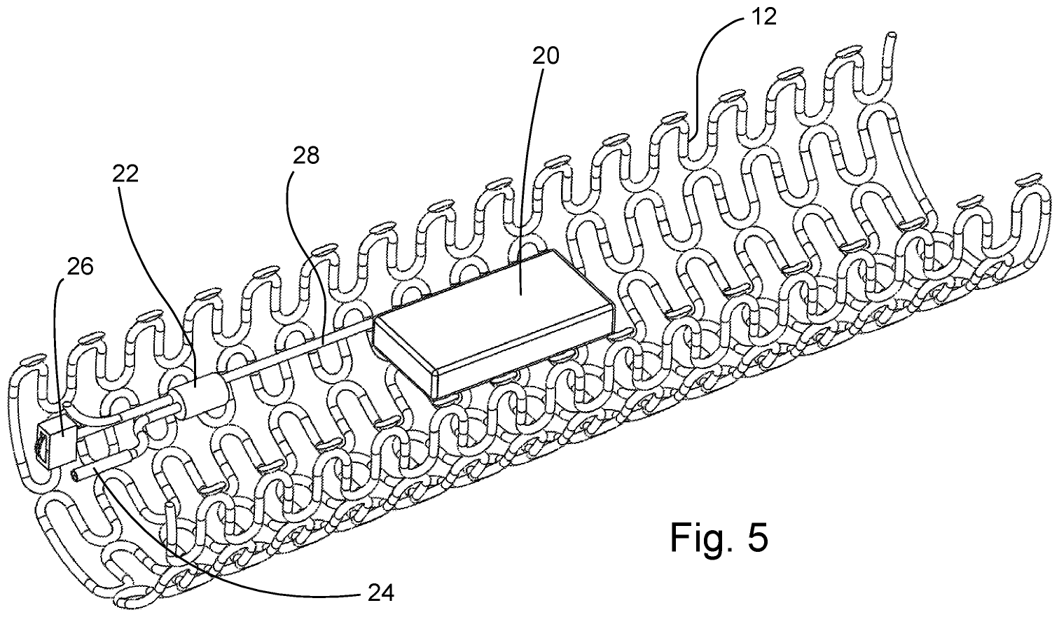

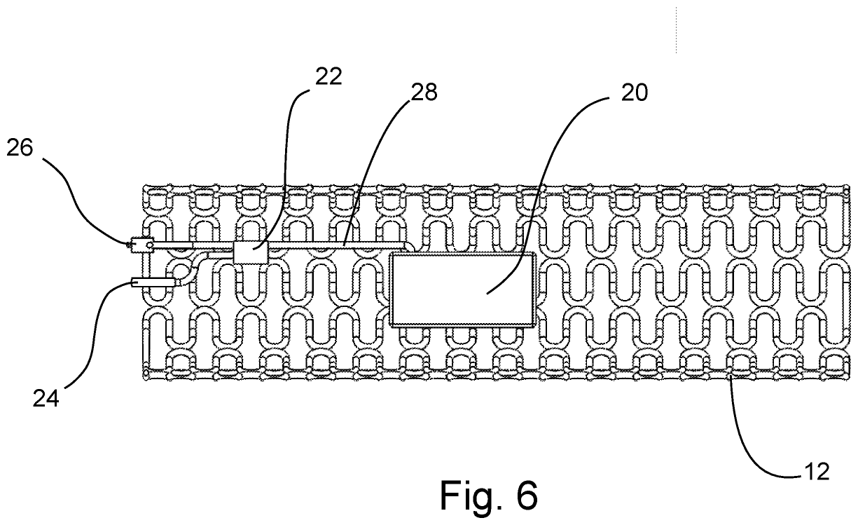

FIG. 4 is an isometric view of the internal heating grid 12 with electrical components the layout of the electrical heating components. The figure is shown without any of the isolative layers for a conceptual understanding on how the internal components are disposed within the embodiment. The thermal management control 26 is shown to be wired to a junction connection 22 by way of internal wires 28. The connection also connects the power port 24, battery 20, and heating grid 12, wherein the junction connection 22 may further include a control module, such as a circuit board, for more complex configurations, or a simple wiring junction for embodiments, such as the embodiment shown in FIG. 4. FIG. 5 is an enlarged isometric cut-view of the internal heating grid with electrical components shown in FIG. 4, and FIG. 6 is an enlarged side cut-view of the internal heating grid with electrical components shown in FIG. 4. FIGS. 5 and 6 may be appreciated to have the same elements as FIG. 4, but is illustrated to a higher degree of clarity in regards to location of the elements.

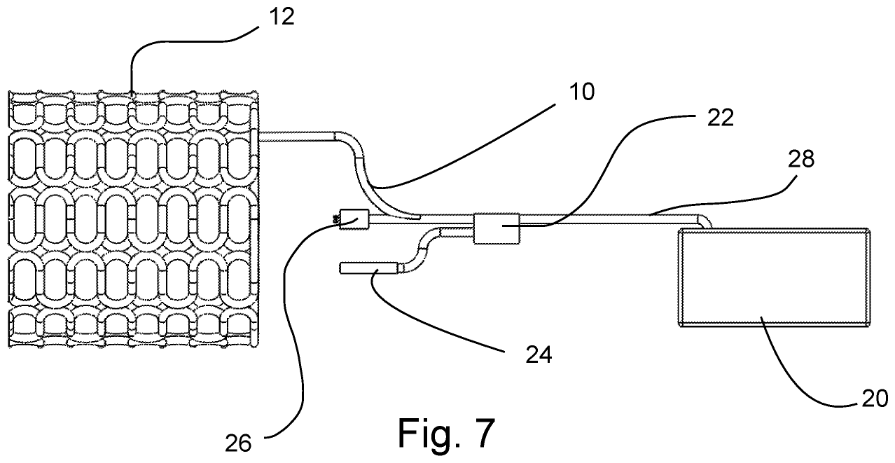

FIG. 7 is an exploded conceptual view of the electrical components of the system. Though the layout of the electrical components are not in their exact locations, as shown in FIGS. 4-6, FIG. 7 shows the electrical components spread out for clarity. In the figure may be seen the heating element 12, connected by a heating element interface wire 10, connected to a connection junction 22, which connects the variable thermal management control 26, power port 24, and connects the battery 20 by internal wiring 28.

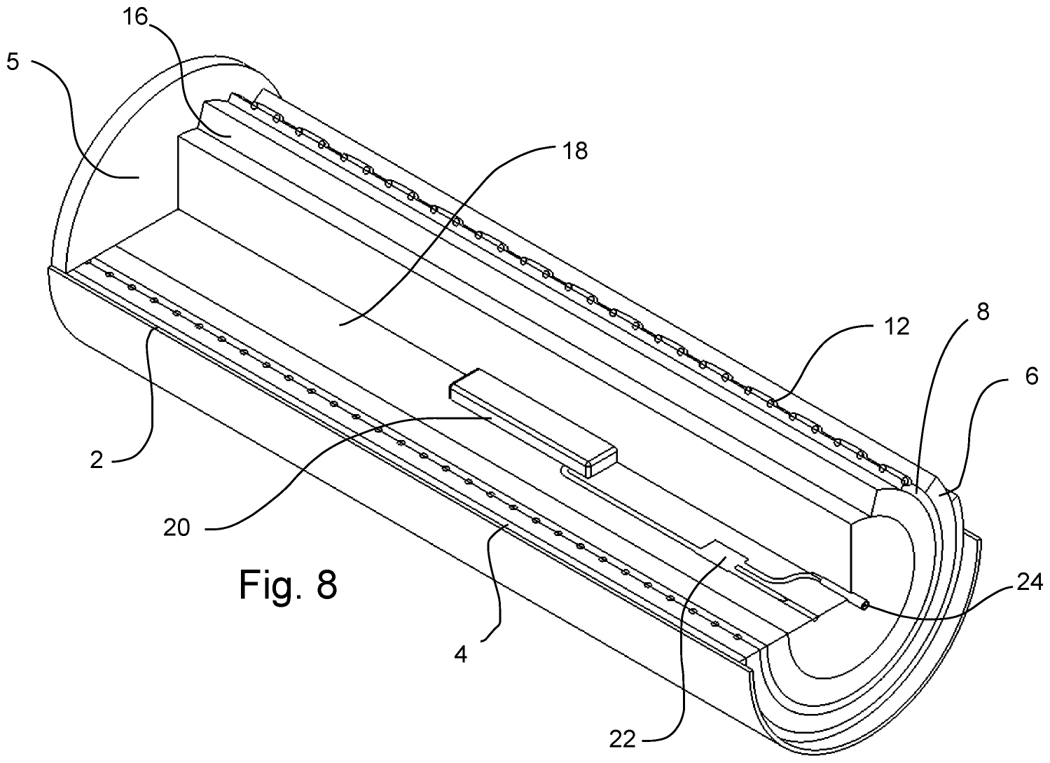

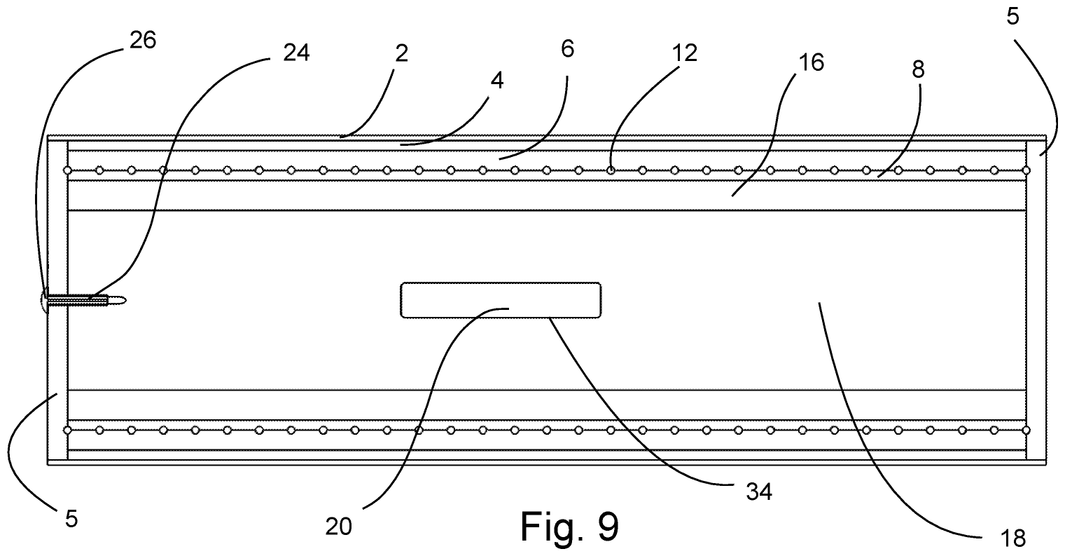

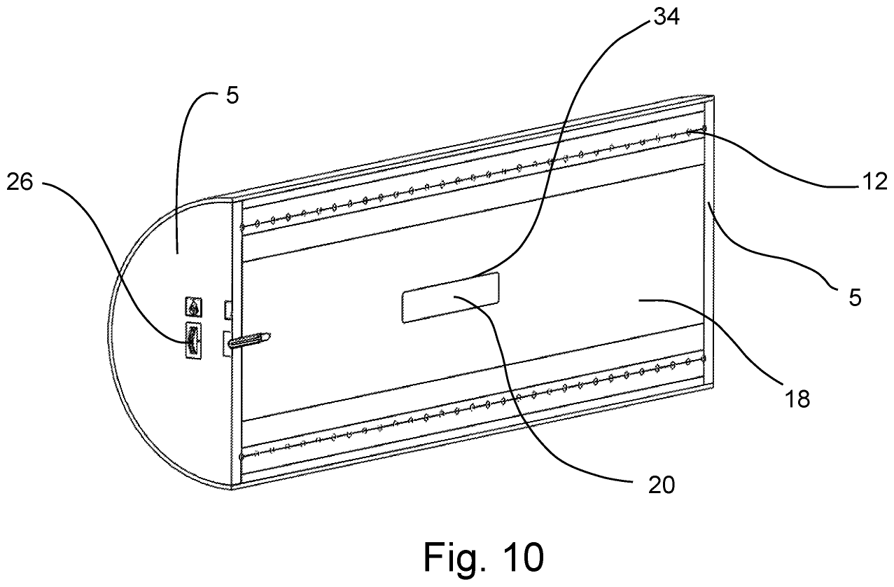

FIG. 8 is an isometric cut-view of the thermal-electronic massage roller 100, FIG. 9 is a side-cut view of the thermal-electronic massage roller 100, and FIG. 10 is an isometric side-cut view of the thermal-electronic massage roller 100. FIGS. 8-10 show the internal layers of the solid foam roller 100. As may be appreciated, an outermost layer is the washable sleeve 2. Internal to the washable sleeve 2 is the conductive sleeve 6, wherein the conductive sleeve 6 is configured to evenly distribute heat from the heating element 12 in the heating element binder layer 8. Under the heating element binder layer are the insulative layer 16, and the inner core layer 18, wherein the electrical components, including the battery 20, wiring, 28, control means 26, and power port 24 are safely insulated from heat.

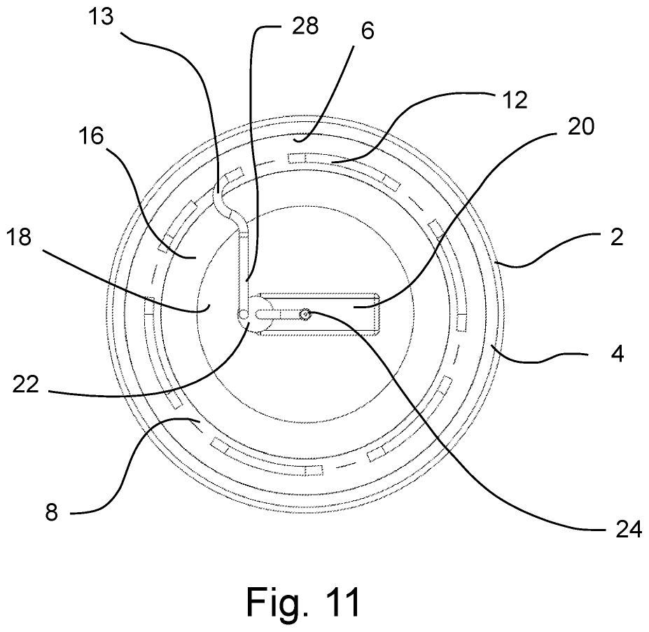

FIG. 11 is a front cut view of the thermal-electronic massage roller showing internal components, and the layers therein. As may be seen, the battery 20 is in the center of the roller 100. The core 18 surrounds the battery, and the wiring 28, including the wiring 13 for the heating element 12. As may be further appreciated, an insulative layer 16 surrounds the core 18. The insulative layer 16 separates the heating element binder 8 from the core 18, providing a thermal buffer therein. The heating element binder 8 may be seen to include a heating element 12 within. The conductive sleeve 6 can then be seen outside the heating element binder 8, wherein it is in thermal communication with the removable sleeve layer 2.

An embodiment of the invention provides for a heated massage roller 100. The heated massage roller 100 is a device with a cylindrical body having at least six layers, wherein the at least six layers contact one another and form a generally solid body. End caps 5 securely contain the internal components to the heated massage roller 100.

An innermost core layer 18 is formed of a substantially solid insulative material with a plurality of cavities 34 for internal components of the device 100. The internal components include a battery 20, a power port 24 in electrical communication with the battery 20, and a thermal control module 26 in electrical communication with the battery 20. The innermost layer 18 includes and an electronic couple between the heating element 12 and the battery 20. An insulative layer 16 is included that cylindrically surrounds the innermost core layer 18, providing a thermal buffer between the innermost core layer 18 and a heating element binder layer 8. The heating element binder layer 8 cylindrically surrounds the insulative layer 16. The heating element 12 is set within the heating element binder layer 8. A conductive sleeve 6 cylindrically surrounds the heating element binder 8, and the conductive sleeve 6 is constructed of conductive material capable of pulling heat from the heating element 12 in an outward direction away from the core 18 of the device. A stiff cylindrical layer 4 is included and surrounds the conductive sleeve 6 to provide the structure for the device. An outermost layer 2 surrounds the stiff cylindrical layer 4 and is configured to be a formable material, wherein the outermost layer is removable.

The stiff cylindrical layer 4 includes textured elements to further assist in massage, wherein the textured elements allow for better grip of the device and increase pressure received by a user in targeted areas. These may include a ribbed texture or studded texture, but other suitable protrusions may also be implemented in other embodiments. In some embodiments, the stiff cylindrical layer 4 is constructed from a rubber, a thermoset, a dense foam of neoprene, EVA, or combination of these materials, and configured to be hydrophobic to prevent penetration of moisture.

In some embodiments, the innermost core layer 18 utilizes a split-shell design, wherein the innermost core 18 is divided into at least two separate portions to allow easier access to nested electronic components 10/20/22/24/26/28 by dividing the core in to two halves, separated at the core's diameter, to expose the nesting cavities 34. This core layer 18 may be formed from a moldable foam of polyurethane or silicone.

In some embodiments the insulative layer 16 is comprised of a material including a closed cell foam polyethylene. In some embodiments, the heating element binder layer 8 is comprised of a dense potting material, which may be a silicone, epoxy, or urethane. In some embodiments, the heating element 12 is a metallic cylindrical grid that, when electrically engaged, is capable of variable heating levels, and is constructed of a metallic resistive conductor material. In some embodiments, the heating element 12 further comprises a doped ferrous metallic. In some embodiments, the conductive sleeve 6 is constructed of material, wherein the material is at least one of silver, copper, aluminum, or ferrous metals, and is configured in a metallic matrix. In some embodiments, the outermost layer 2 is further comprised of an antimicrobial, removable, and washable sleeve constructed of a soft fabric, which is configured to conform to geometric properties of the stiff cylindrical layer. The soft fabric may be a cotton cloth, a polyester cloth, or a rayon cloth.

In some embodiments, the thermal control module 26 is a variable dial with a plurality of heating levels 30, including an off level 32. In other embodiments, the thermal control module 26 is a on/off engagement including at least one of a switch or a button.

An embodiment of the invention provides for a method for configuring a massage roller 100 for thermal transfer. This method includes configuring a device 100 in a multi-layer construction, wherein the device includes at least six layers.

The method further includes configuring each layer in to perform a specific function, wherein an outermost layer 2 is configured to be a removable and washable layer that comprises form-fitting properties of a layer immediately beneath the outermost layer, a layer beneath an outermost layer is configured to be a stiff cylindrical layer 4 to give the device structure and shape, a conductive sleeve layer 6 is configured to be beneath the stiff cylindrical layer 4 to evenly distribute heat of a heating element 12, a heating element binder layer 8 is configured to be beneath the conductive sleeve 6 layer to contain the heating element 12 and provide a buffer between levels beneath the heating element binder layer 8, configuring a layer beneath the heating element binder layer 8 to be an insulative layer 16 to provide a heat buffer between the heating element 12 and a core structure 18 containing electronic components 10/20/22/24/26/28, and configuring the core structure 18 as an innermost layer to contain the electronic components 10/20/22/24/26/28 and act as one final heat buffer between the heating element 12 and the electronic components 10/20/22/24/26/28, and to contain the electronic components 10/20/22/24/26/28 from movement by nesting the electronic components 10/20/22/24/26/28 in a solid material.

The method further includes configuring the conductive sleeve layer 6 to draw heat away from the core 18 by using conductive material to draw heat in a singular direction and thermally transfer the heat to a user's body by implementing a metallic matrix to act as a heat sync to transfer heat from the heating element 12 to the user's body, and configuring the heating element 12 for uniform heat transfer by implementing a cylindrical grid 12 of conductive material, that, when electrically engaged, is capable of variable heat output.

To allow a user to control the device 100, the method includes configuring the device 100 to include a variable heat control interface 26, wherein the level of heat may be adjusted to suit a user's preference by including a variable dial having a plurality of heating levels 30 and an off setting 32.

The method also includes steps to optimize the functionality of the device by implementing different materials. The method includes configuring the outermost layer 2 for use as a removable and washable layer by constructing the outermost layer 2 from a cotton cloth, a polyester cloth, or a rayon cloth, configuring the stiff cylindrical layer 4 for optimal strength and structure by constructing the stiff cylindrical layer 4 from a rubber, a thermoset, a dense foam of neoprene, or EVA, configuring the conductive sleeve 6 for even thermal transfer by constructing the conductive sleeve 6 from silver, copper, aluminum, or ferrous metals, configuring the heating element binder layer 8 for optimal thermal performance by constructing the heating element binder layer 8 from silicone, epoxy, or urethane, configuring the insulative layer 16 for optimal thermal buffering by constructing the insulative layer 16 from a closed cell foam polyethylene, configuring the core structure 18 for optimal support and thermal buffer by constructing the core structure 18 from a moldable foam of polyurethane, or silicone, and configuring the heating element for optimal heat production and thermal transfer by constructing the heating element 12 from a doped ferrous metallic.

The exact materials and configurations are not limiting to the invention, and the elements of the embodiment may include the referenced materials, but are not limited to only those materials, as suitable alternatives may also be appropriate.

While there has been shown and described above the preferred embodiment of the instant invention it is to be appreciated that the invention may be embodied otherwise than is herein specifically shown and described and that, within said embodiment, certain changes may be made in the form and arrangement of the parts without departing from the underlying ideas or principles of this invention as set forth in the Claims appended herewith.

I claim:

1. A thermal electronic massage roller, comprising:
a device with a cylindrical body comprising at least six layers, wherein said at least six layers contact one another and form a generally solid body;
an innermost core layer, of said at least six layers, formed of a substantially solid insulative material with a plurality of cavities for internal components of said device, wherein said internal components include at least a battery, a power port in electrical communication with said battery, and a thermal control module in electrical communication with said battery;
an insulative layer, of said at least six layers, cylindrically surrounding said innermost core layer, providing a thermal buffer between said innermost core layer and a heating element binder layer;
said heating element binder layer, of said at least six layers, cylindrically surrounding said insulative layer;
a heating element set within said heating element binder layer;
a conductive sleeve cylindrically surrounding said heating element binder, wherein said conductive sleeve is constructed of conductive material capable of pulling heat from the heating element in an outward direction away from the core of the device;
a stiff cylindrical layer, of said at least six layers, surrounding the conductive sleeve to provide the structure for the device;
an outermost layer, of said at least six layers, surrounding said stiff cylindrical layer is configured to be a formable material, wherein the outermost layer is removable; and
said innermost layer, of said at least six layers, further includes and an electronic couple between said heating element and said battery.

2. The heated massage roller as recited in claim 1, wherein the stiff cylindrical layer includes textured elements to further assist in massage, wherein the textured elements allow for better grip of the device and increase pressure received by a user in targeted areas.

3. The heated massage roller as recited in claim 2, wherein the textured elements include at least one of a ribbed texture or studded texture.

4. The heated massage roller as recited in claim 1, wherein the stiff cylindrical layer is constructed from at least one of a rubber, a thermoset, a dense foam of neoprene, or EVA, and configured to be hydrophobic to prevent penetration of moisture.

5. The heated massage roller as recited in claim 1, wherein said innermost core layer utilizes a split-shell design, wherein said innermost core is divided into at least two separate portions to allow easier access to nested electronic components.

6. The heated massage roller as recited in claim 1, wherein said innermost core layer is comprised of a material of at least one of a moldable foam of polyurethane or silicone.

7. The heated massage roller as recited in claim 1, wherein said insulative layer is comprised of a material including a closed cell foam polyethylene.

8. The heated massage roller as recited in claim 1, wherein said heating element binder layer is comprised of a dense potting material.

9. The heated massage roller as recited in claim 8, wherein said dense potting material is at least one of a silicone, epoxy, or urethane.

10. The heated massage roller as recited in claim 1, wherein said heating element is a metallic cylindrical grid that, when electrically engaged, is capable of variable heating levels, and is constructed of a metallic resistive conductor material.

11. The heated massage roller as recited in claim 10, wherein said heating element further comprises a doped ferrous metallic.

12. The heated massage roller as recited in claim 1, wherein said conductive sleeve is constructed of material, wherein said material is at least one of silver, copper, aluminum, or ferrous metals, and is configured in a metallic matrix.

13. The heated massage roller as recited in claim 1, wherein said outermost layer is further comprised of an antimicrobial, removable, and washable sleeve constructed of a soft fabric, which is configured to conform to geometric properties of the stiff cylindrical layer.

14. The heated massage roller as recited in claim 13, wherein said soft fabric is at least one of a cotton cloth, a polyester cloth, or a rayon cloth.

15. The heated massage roller as recited in claim 1, wherein said thermal control module is a variable dial with a plurality of heating levels, including an off level.

16. The heated massage roller as recited in claim 1, wherein said thermal control module is a on/off engagement including at least one of a switch or a button.

17. The heated massage roller as recited in claim 1, further comprising: end caps to securely contain the internal components to the heated massage roller.

18. A method for configuring a massage roller for thermal transfer, comprising:
configuring a device in a multi-layer construction, wherein said device includes at least six layers;
configuring each layer in said at least six layers to perform a specific function, wherein an outermost layer is configured to be a removable and washable layer that comprises form-fitting properties of a layer immediately beneath said outermost layer, a layer beneath an outermost layer is configured to be a stiff cylindrical layer to give the device structure and shape, a conductive sleeve layer is configured to be beneath the stiff cylindrical layer to evenly distribute heat of a heating element, a heating element binder layer is configured to be beneath the conductive sleeve layer to contain the heating element and provide a buffer between levels beneath the heating element binder layer, configuring a layer beneath the heating element binder layer to be an insulative layer to provide a heat buffer between the heating element and a core structure containing electronic components, and configuring said core structure as an innermost layer to contain the electronic components and act as one final heat buffer between the heating element and the electronic components, and to contain said electronic components from movement by nesting said electronic components in a solid material;
configuring the conductive sleeve layer to draw heat away from the core by using conductive material to draw heat in a singular direction and thermally transfer the heat to a user's body by implementing a metallic matrix to act as a heat sync to transfer heat from the heating element to the user's body; and
configuring said heating element for uniform heat transfer by implementing a cylindrical grid of conductive material, that, when electrically engaged, is capable of variable heat output.

19. The method for configuring a massage roller for thermal transfer, as recited in claim 18, further comprising:
configuring the device to include a variable heat control interface, wherein the level of heat may be adjusted to suit a user's preference by including a variable dial having a plurality of heating levels and an off setting.

20. The method for configuring a massage roller for thermal transfer, as recited in claim 19, further comprising:
configuring the outermost layer for use as a removable and washable layer by constructing the outermost layer from a material chosen from the group consisting of a cotton cloth, a polyester cloth, and a rayon cloth;
configuring the stiff cylindrical layer for optimal strength and structure by constructing the stiff cylindrical layer from a material chosen from the group consisting of a rubber, a thermoset, a dense foam of neoprene, and EVA;
configuring the conductive sleeve for even thermal transfer by constructing the conductive sleeve from a material chosen from the group consisting of silver, copper, aluminum, or ferrous metals;
configuring the heating element binder layer for optimal thermal performance by constructing the heating element binder layer from a material chosen from the group consisting of silicone, epoxy, and urethane;
configuring the insulative layer for optimal thermal buffering by constructing the insulative layer from a closed cell foam polyethylene;
configuring the core structure for optimal support and thermal buffer by constructing the core structure from a material chosen from the group consisting of a moldable foam of polyurethane, and silicone; and
configuring the heating element for optimal heat production and thermal transfer by constructing the heating element from a doped ferrous metallic.
